(19) United States
(12) Patent Application Publication
(10) Pub. No.: US 2022/0178629 A1
(43) Pub. Date: Jun. 9, 2022

(54) SYSTEM AND METHOD OF EVAPORATIVE COOLING WATER MANAGEMENT

(71) Applicant: FTD Solutions LLC, Colorado Springs, CO (US)

(72) Inventors: Boris Eliosov, St. Marys, PA (US); Vyacheslav Libman, Danville, CA (US)

(21) Appl. No.: 17/541,496

(22) Filed: Dec. 3, 2021

Related U.S. Application Data
(60) Provisional application No. 63/121,607, filed on Dec. 4, 2020.

(51) Int. Cl. F28F 19/01; F28D 5/02; G01N 27/06; C02F 1/68; C02F 1/50; C02F 1/00; G01N 33/18; F28F 19/00; G05B 19/042; C02F 103/02

(52) U.S. Cl. CPC F28F 19/01; C02F 2301/046; G01N 27/06; C02F 1/68; C02F 1/50; C02F 1/001; G01N 33/1826; F28F 19/00; G05B 19/042; C02F 1/008; C02F 2209/05; C02F 2103/023; C02F 2209/005; C02F 2303/08; C02F 2303/04; C02F 2209/11; C02F 2209/20; C02F 2209/06; G05B 2219/2605; C02F 2209/01; C02F 2303/16; C02F 2303/22; C02F 2209/04; F28D 5/02

(57) ABSTRACT

A system and method of managing water in an evaporative cooling system includes one or a combination of three components. A first component is to purify incoming water to the target quality. A second component is to purify condenser loop water to the target quality. The choice of the technology may depend on the site specific environmental conditions determining the quality of the recirculated condenser water and may include (but not limited to) filtration allowing for meeting target quality parameters, such as ultrafiltration, microfiltration, etc. A third component is to provide protection for the condenser loop hardware, preventing or reducing rate of corrosion, fouling, and scaling by adding chemicals to water in the system. The choice of the chemistry will depend on the site specific environmental and other system operating conditions

### CROSS REFERENCE TO RELATED APPLICATIONS

This application is a claims benefit of and is a Non-Provisional of U.S. Provisional Application Ser. No. 63/121,607, filed Dec. 4, 2020, pending, which application is hereby incorporated by this reference in its entirety.

### BACKGROUND

### Field

Embodiments of the present invention relate to evaporative cooling systems, specifically a system and a method of evaporative cooling system water management.

### Background

Condenser loop systems are intended to dissipate heat generated by mechanical equipment. In evaporative cooling systems, the heat dissipation is achieved by water evaporation. Water evaporation results in raising concentration of chemicals originated from the make-up water, such as metals, chlorides, organics, and alkalinity. In order to prevent oversaturation, some of the water (blowdown) is bled from the system and replaced with the make-up water. This results in loss of water, as well as expensive treatment chemicals that are added to prevent corrosion, scaling, and biological growth, and environmental impacts from chemicals in the waste stream.

U.S. Pat. No. 4,475,356 discloses a method for controlling the blowdown flowrate using a temperature probe that is placed in the recirculating water. The signal from the temperature probe controls the blowdown valve position so that when recirculating water temperature rises, more of the recirculating water is blown down. Chemical addition and make-up water systems are controlled to compensate for the increased blowdown.

U.S. Pat. No. 5,294,916 discloses a blowdown control method that is based on electrical conductivity measurement. An electrical conductivity sensor controls the blowdown valve in the way that the valve opens when the conductivity exceeds a predetermined threshold and closes when the conductivity value is below the threshold. The water is replenished with a make-up water to maintain the constant volume of water in the system.

U.S. Pat. No. 5,730,879 method for controlling alkalinity in cooling towers without addition of strong acids. The method is based on passing a controlled side stream through a strong cation exchange media. The flowrate of the side stream is controlled in the way to ensure pH of the recirculating water is maintained at the predetermined level of the saturation pH. The method is claimed to reduce blowdown and consequently, make-up water consumption.

The inventions described above are intended to ensure the recirculating water parameters are maintained in the range that ensures reliable operation and in combination with addition of appropriate treatment chemicals minimizes risks related to scaling, corrosion, and biofouling. In all the above processes, constant addition of treatment chemicals is required to maintain their concentration at the required range. The rising importance of water conservation and high cost of water treatment chemicals used in condenser loop systems has made blowdown reduction a target for performance optimization.

International patent application WO/2004/028978 A1 discloses the method of minimizing cooling towers blowdown as the result of the softening of the make-up water in combination with acid addition and drift control. The key element for minimizing the blowdown is make-up water softening. According to the publication, softening potentially achieves two goals: removal of scaling cations and alkalinity reduction if cations are replaced with hydrogen ions. Alternatively, alkalinity reduction can be achieved by acid addition to the system.

US patent application US2009/0211983 A1 discloses method of reducing the blowdown by softening all make-up water using sodium-based cation exchange in combination with addition of treatment chemicals to control scaling, corrosion, and biofouling. Ion exchange softening is required for hardness reduction to 10 mg/L as calcium carbonate. According to the publication, the method for controlling the blowdown may include:
Sodium cation exchange softening
Bypass filtration to remove suspended solids from the cooling water
Chemical treatment to control scaling and corrosion
Electrolytic bromine as a biocide

U.S. Pat. No. 9,315,396 B2 discloses the method of utilizing reverse osmosis (RO) reject as a make-up water in cooling towers. The method includes hardness removal from the RO reject, silica adjustment in the treated reject to ensure at least 200 mg/L of SiO2 is maintained in recirculating water and maintaining pH 9 in recirculating water. Additional water conservation is achieved by recovery some of the blowdown water using a forward osmosis (FO) process.

All the above disclosures for minimization of the blowdown flowrate rely on hardness removal and alkalinity reduction. However, hardness and alkalinity are not the only parameters that determine the blowdown. Although in many cases they are indeed the main scaling contributors, other chemicals can limit the benefits of softening for blowdown minimization. Examples of such chemicals are silica, which can result in fouling when oversaturated, and chlorides that must be controlled to prevent excessive corrosion. As the result, the above disclosures cannot substantially eliminate the blowdown.

### BRIEF SUMMARY OF THE DISCLOSURE

Accordingly, the present invention is directed to a system and a method of evaporative cooling water management that obviates one or more of the problems due to limitations and disadvantages of the related art.

The systems and methods disclosed in this application provide a method of evaporative cooling system water management that is intended to overcome the limitations of the current state-of-the-art and significantly reduce operational cost of condenser loop systems. This is achieved by supplying the system with a high quality feedwater and treating the recirculating water via a sidestream to maintain a high level of water quality. Cost reduction is the result of reduced make-up water consumption and eliminating the need for continuous treatment chemical addition. Potential users of this application include industrial clients and multistory buildings.

It is to be understood that both the foregoing general description and the following detailed description are exemplary and explanatory only and are not restrictive of the invention as claimed.

### BRIEF DESCRIPTION OF THE DRAWINGS

The accompanying figures, which are incorporated herein and form part of the specification, illustrate the system and the method of evaporative cooling water management. Together with the description, the figures further serve to explain the principles of the system and method of evaporative cooling water management described herein and thereby enable a person skilled in the pertinent art to make and use the system and method of evaporative cooling water management.

FIG. 1 illustrates an example of a generic evaporative cooling system.

FIG. 2 illustrates an example of an evaporative cooling system.

FIG. 3 illustrates components of an evaporative cooling water management system according to principles described herein.

### DETAILED DESCRIPTION

Reference will now be made in detail to embodiments of the system and the method of evaporative cooling water management with reference to the accompanying figures. The same reference numbers in different drawings may identify the same or similar elements.

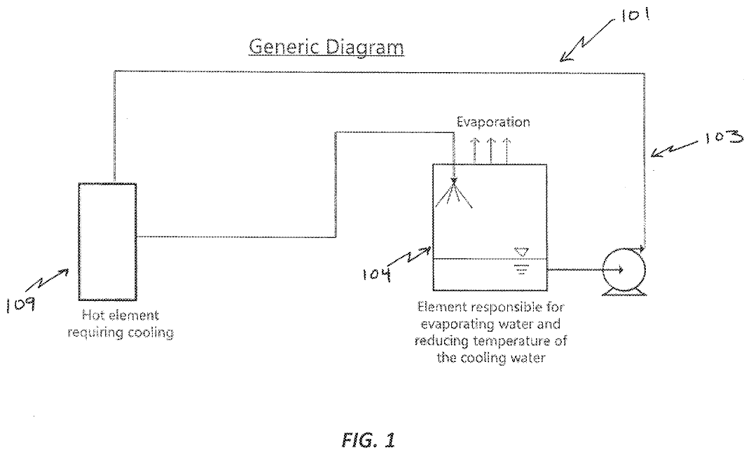

FIG. 1 illustrates a generic system 101 for evaporative cooling in which water is circulated in a “condenser loop” 103 between an evaporative cooling element 104 and a hot element 109, which can be any element requiring cooling. The evaporative cooling element 104 is responsible for evaporating water and reducing temperature of the cooling water, which is then returned to the hot element. The present disclosure is not limited by the element that requires cooling nor is it intended to be limited by the structure of the evaporative cooling system.

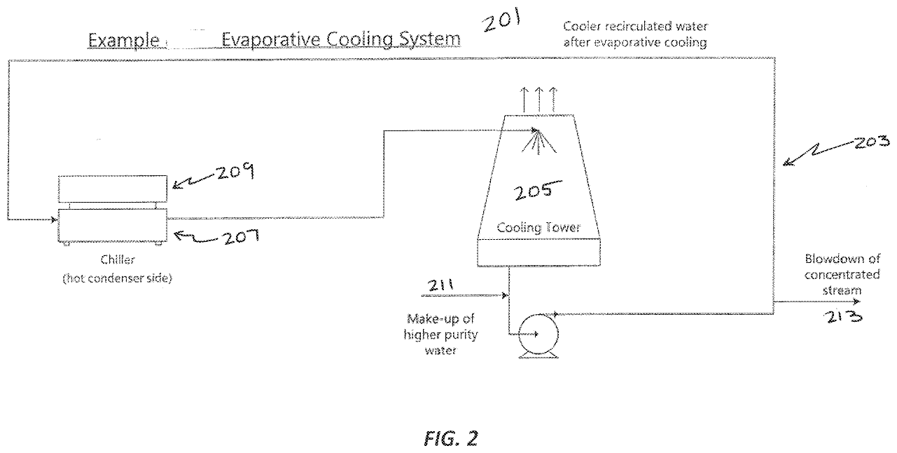

FIG. 2 illustrates an example evaporative cooling system 201 with blowdown for impurity removal and addition of high purity make-up water for continuation of evaporative cooling, in addition to recirculation of water within the system. As shown, an example evaporative cooling system 201 may include a cooling tower 205 and a chiller 207, and a recirculating water flow path 203, e.g., pipes, between the chiller and the cooling tower 205 whereby the cooling tower 205 supplies water of at a temperature less than that of a hot element 209 to be cooled. The hot element 209 is in thermal communication with the chiller 207 for heat exchange between the hot element 209 and the water from the cooling tower such that water of increased temperature is returned to the cooling tower 205 via a water recirculation path 203. Heat is removed from water in the cooling tower 205 by evaporation, resulting in a loss of the amount of water in the cooling tower 205, which is supplemented by make-up water 211. The amount of water in the cooling tower 205 is further reduced by blowdown 213 to remove concentrated contaminants from the cooling tower, and water in the cooling tower 205 may be supplemented by make-up water. Examples of evaporative cooling systems/condenser loops are provided at https://bigladdersoftware.com/epx/docs/8-6/plant-application-guide/condenser-loop-condenser-loop-cooling-tower.html, disclosure of which is hereby incorporated by references for all purposes as if fully set forth herein.

In an example condenser loop provided at https://bigladdersoftware.com/epx/docs/8-6/plant-application-guide/condenser-loop-condenser-loop-cooling-tower.html, a cooling tower 305 and a pump 315 may supply cooling water to a chiller 307, which may be electric. Therefore, the supply side of the loop contains the cooling tower 305 and the demand side contains the chiller 307. The loop is operated by using plant equipment operation schemes, and schedules. Main components on the supply side half loop for the Condenser Loop are the Cooling Tower 305 that supplies the cooling water and the constant speed pump 315 that circulates the cooling water through the loop, which includes condenser side water supply line 317. This half loop supplies cooling water to the electric chiller 317 on the demand side half loop. The main component on the demand side half loop is the chiller 307 that uses the cooling water supplied by the cooling tower 307. The chiller 307 in turn is used to supply chilled water in a primary cooling loop (not shown).

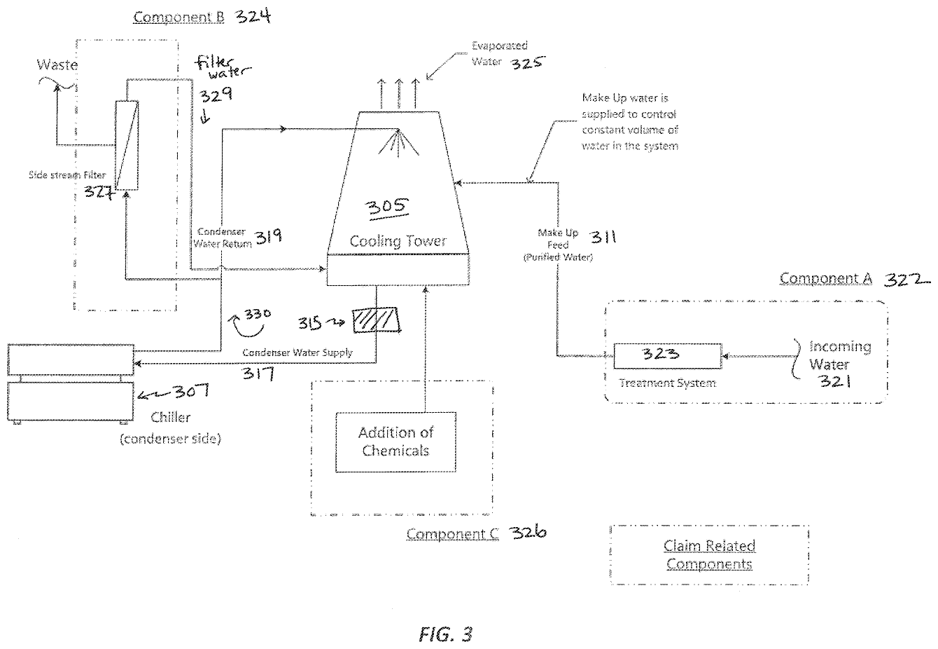

According to principles described herein performance of an evaporative water-cooling system 301 may be improved via management of the water within the system. Referring to FIG. 3, a system for water management in an evaporative cooling system according to principles described herein includes a high quality water source 322 (Component A), a side stream filter 324 (Component B) and/or a chemical additive source 326 (Component C). The use of these three components in a condenser loop substantially reduces or eliminates the need for blowdown to remove contaminants. By evaporating water without blowdown or with only “unintended” blowdown, the feed water parameter concentration is, in theory, increased indefinitely. In reality, there are “drift” losses in the form of small droplets out of the cooling towers. The feed water quality may be calculated based on those losses, which becomes “unintended blowdown.”

Accordingly, subsystems/components A, B and/or C are used in conjunction with an evaporative cooling system, non-limiting examples of which are shown in FIGS. 1 and 2.

According to an aspect of the present disclosure, the feed water/make up water should contain a very low amount of organic and inorganic contamination and the recirculating water needs to be filtered to remove contaminants that may be contributed by the air or piping in the system. Referring to Component A of FIG. 3, feed water 321 is provided to a cooling tower 305 of an evaporative cooling system 301. The cooling tower 305 may be any cooling tower configuration for evaporative cooling. The feed water 321 can originate from any system providing appropriate, high purity water, for example, from a dedicated make up water treatment system, from any water reclamation system that produces high quality water, from water effluent from certain operations that utilize high quality water, such as for example ultra-pure water used by semiconductor and pharmaceutical factories, or from any method/source that produces high quality water. For example, make up water can be reverse osmosis (RO) product water obtained from dedicated feed water treatment systems, RO product water obtained from a water reclamation system, or RO product water obtained from effluent of operations that utilize RO quality water.

Initially, feed water 321 may be supplemented with anions and cations for corrosion protection such as Calcium and alkalinity, in the amount to maintain them in the optimal operating range. Additional initial supplement may be treatment chemicals for corrosion protection, scaling prevention, and biocides. Calcium and alkalinity may be initially supplemented to maintain their concentration in the system after evaporation in a predetermined range. For example, but not limited to, a range of 50-500 mg/L and 50-250 mg/L as CaCO₃, respectively. RO reject may be used to supplement Calcium and alkalinity. For this purpose, there may be a treatment system between the incoming water input and the cooling tower 305.

During the system operation, water evaporation is compensated with the high-quality feed/make up water 311 with no addition of any chemicals as the chemicals that were initially added to the feed water 321 will remain in the system. That is, substantially constant water volume is maintained throughout the evaporative cooling system. Since the feed water 321 contains a very low amount of organic and inorganic contamination, concentration of all chemicals in the recirculating water will remain constant. In fact, with respect to water chemical treatment, the system will be operated in a manner similar to a “closed-loop” system such as for example, process cooling water (PCW).

Preventing accumulation of suspended solids in the system is achieved by filtration of a side stream of recirculating water as illustrated in FIG. 3, Component B. As illustrated, water from a chiller 307 in the evaporative cooling system 307, the chiller 307 for removing heat from an element requiring heat reduction, is at least partially filtered by a side stream filter 327 before being returned to the cooling tower 305. That is, at least a portion of the recirculating water is diverted from a main flow path 330 to a filter 327 to remove suspended solids. The filter 327 may be of any appropriate type for removing suspended solids and diverting waste from the diverted water stream. The filtered water 329 is then returned to the cooling tower 305, either directly or by adding the filter water output stream to a main recirculation stream 319 between the chiller 307 and the cooling tower 305. In some aspects all water from the chiller plant 307 may be passed through the side stream filter 327. It is contemplated herein that the side stream filter 327 is on the “hot side” of the water circulation path (e.g. condenser water return path 319), but placement of the side stream filter 327 may be at any location within the recirculation path 330.

In other words, a system and method of managing water in an evaporative cooling system includes one or a combination of three components. A first component is to purify incoming water to the target quality. In any of the disclosed embodiments, the choice of water treatment technology may depend on the quality of the treated feedwater and may include one or combination of continuous deionization, nanofiltration, reverse osmosis, or equivalent technologies supporting the purified water quality requirements. A second component is to purify condenser loop water to the target quality described in the claim. In any of the disclosed embodiments, the choice of the technology may depend on the site specific conditions determining the quality of the recirculated condenser water and may include (but not limited to) filtration allowing for meeting target quality parameters, such as ultrafiltration, microfiltration, etc. A third component is to provide protection for the condenser loop hardware, preventing or reducing rate of corrosion, fouling, and scaling by adding chemicals to water in the system. In any of the disclosed embodiments, the choice of the chemistry will depend on the site specific environmental and other system operating conditions. Corrosion inhibitors must be stable over targeted residence time in the system. According to the principles of the substantially “closed loop” water management system described herein, the need for intentional blowdown is substantially reduced or eliminated as compared to conventional evaporative cooling systems. Thus, chemicals used to treat water in the evaporative cooling system are able to be retained in the system because they are not lost in the blowdown.

It will be apparent to those skilled in the art that various modifications and variations can be made in the present invention without departing from the spirit or scope of the invention.

In an aspect, a method of water management in an evaporative cooling system comprising: a high temperature element requiring cooling; an evaporative system and water based coolant recirculating between the two includes providing an initial portion of water at a first amount and quality to the evaporative system, wherein the water contains organic and inorganic contamination of less than 20 ppb total organic carbon (TOC) and/or wherein the water contains conductive ions in a concentration resulting in resistivity equal to or greater than 0.02 MΩ·cm; treating the initial portion of water in the evaporative system with at least one chemical; causing evaporation of the treated make-up water to reduce the first amount of water to a second amount of water in the evaporative cooling system container; and adding make-up water to the evaporative cooling system container to increase volume of the water in the container from the second amount to the first amount without discarding substantially any of the treated fill water. The high temperature element may be on an evaporator side of the chiller or other heat source. The evaporative cooling system may be on a condenser side of the chiller or other heat source. The chemical may be a corrosion inhibitor, a biocide, an antiscalant and/or a dispersant.

In an aspect, an apparatus for evaporative cooling system water management, the apparatus includes a evaporative cooling system, a heat source and a water recirculation loop fluidically coupling the evaporative cooling system and the heat source such that the heat source is capable of receiving cooling water supply from the evaporative cooling system via the recirculation loop; a purified water feed system having an incoming water inlet for receiving water and fluidic ally coupled to the evaporative cooling system to provide the water to the evaporative cooling system; a chemical treatment system fluidically coupled to the evaporative cooling system to provide at least one of corrosion inhibitors, biocides, antiscalants and/or dispersants to water in the cooling tower; and a water filtration system comprising a side stream or full stream filter fluidically coupled to an output of the heat source and an input of the evaporative cooling system, for filtering water from the heat source to remove waste and provide the filtered water to the cooling tower. The apparatus may include a pump and/or a valve. The heat source may be fluidically coupled to the evaporative cooling system such that a portion of the water output by the heat source bypasses the water filtration system. In some aspects the first water quality is organic and inorganic contamination of less than 20 ppb TOC and/or the fill water contains conductive ions in a concentration resulting in resistivity equal to or greater than 0.02 MΩ·cm. The evaporative cooling system may include a pressurized feedline, and may include one of or a combination of a pump and valve. The system may include sensors for sensing at least one of conductivity, oxidation-reduction potential (ORP), pH, tracer concentration, silt density index (SDI), turbidity, TOC, and resistivity. For example, a sensor or sensors may be in the purified water feed line for measuring TOC and/or resistivity and/or a sensor or sensors may be in the water filtration system for measuring SDI and/or turbidity, and/or a sensor or sensors may be in the chemical treatment system for measuring conductivity, ORP, and/or pH.

In an aspect, an apparatus for treating recirculating water in an evaporative cooling system having a recirculating water condenser loop and side stream includes at least one controller is configured to: direct ingress of a portion of recirculating water from the recirculating water condenser loop flow via the side stream; direct side stream water through the filter to remove contaminants such that the water meets quality criteria of <5 SDI units and turbidity <2 NTU; and direct the filtered side stream water that meets quality criteria to the recirculating water condenser loop.

The apparatus may include at least one of a pump and a valve, wherein the controller if further configured to direct one or a combination of the pump and valve to direct side stream water through the filter and/or direct the filtered side stream water to a recirculating condenser loop.

Any of the systems described herein may include a processor, communicatively coupled to a least one of the purified water feed system, the chemical treatment system and/or the water filtration system for controlling operation of at least of one of the purified water feed system, the chemical treatment system and/or the water filtration system. The processor may be configured to direct ingress of high purity water at a first amount, where water contains a very low amount of organic and inorganic contamination, less than 20 ppb of TOC or equivalent of greater than 0.02 MΩ·cm of conductive ions; direct addition of one or more treatment chemicals that could include: corrosion inhibitors, biocides, antiscalants, or dispersants; direct usage of the water to absorb heat, which water evaporates to a second amount equal or less than the first amount; and direct replenishment of the second amount of water to the first amount without discarding the one or more treatment chemicals that could include: corrosion inhibitors, biocides, antiscalants, or dispersants.

Any of the systems described herein may include a non-transitory computer readable medium for evaporative cooling system water management, configured to execute operations such as directing ingress of water at a first amount to a container, wherein the water contains less than 20 ppb of TOC and/or conductive ions in a concentration resulting in resistivity of greater than or equal to 0.1 MΩ·cm according to sensors measuring one or more of the following: TOC, conductivity; directing addition of one or more treatment chemicals to the water that could include: corrosion inhibitors, biocides, antiscalants, or dispersants, according to sensors measuring one or more of the following: conductivity, ORP, pH; directing usage of the water to absorb heat, which water evaporates to a second amount less than the first amount; and directing replenishment of the second amount of water to the first amount without discarding the one or more treatment chemicals that could include: corrosion inhibitors, biocides, antiscalants, or dispersants.

In another aspect, any of the systems described herein may include a non-transitory computer readable medium for treating recirculating water in an evaporative cooling system having a recirculating water condenser loop and side stream having a filter in a side stream flow path, configured to execute operations such as direct ingress of a portion of recirculating water from the recirculating water condenser loop flow via the side stream; direct side stream water through the filter to remove contaminants such that the water meets quality criteria of <5 SDI units and turbidity <2 NTU; and direct the filtered side stream water that meets quality criteria to the recirculating water condenser loop.

In an aspect as presently disclosed, a method of treating recirculating water in an evaporative cooling system having a condenser loop, includes diverting a portion of recirculating water from the condenser loop flow via a side stream; filtering the diverted portion of recirculating water to remove contaminants such that the water meets quality criteria of <5 SDI units and turbidity <2 NTU; and returning the filtered side stream water that meets quality criteria to the condenser loop flow.

In an aspect, a method of evaporative cooling system water management, may include placing high purity water at a first amount, where water contains a very low amount of organic and inorganic contamination, less than 20 ppb of TOC and conductive ions in concentration that results in resistivity of 0.1 MΩ·cm or higher; adding one or more of treatment chemicals that could include: corrosion inhibitors, biocides, and antiscalants, or dispersants; using water evaporation to dissipate heat resulting in the change of water volume in the system from the first amount to the second amount, which is less than the first amount; and replenishing water to compensate evaporation increasing its volume from the second amount to the first amount without discarding the water containing treatment chemicals.

In an aspect, an apparatus for evaporative cooling system water management, the apparatus comprising at least one controller having circuitry, which one or more controller is configured to: operatively couple to a container or a pressurized line configured to accommodate water; using one or a combination of a pump and valve in case of a pressurized feedline, direct ingress of high purity water at a first amount, where water contains a very low amount of organic and inorganic contamination, less than 20 ppb of TOC or equivalent of greater than 0.02 MΩ·cm of conductive ions; using one or a combination of a pump and valve in case of a pressurized feedline, direct addition of one or more treatment chemicals that could include: corrosion inhibitors, biocides, antiscalants, or dispersants; using a pump, direct usage of the water to absorb heat, which water evaporates to a second amount equal or less than the first amount; and using one or combination of pump or valve in case of a pressurized feedline, direct replenishment of the second amount of water to the first amount without discarding the one or more treatment chemicals that could include: corrosion inhibitors, biocides, antiscalants, or dispersants.

In an aspect, a non-transitory computer readable medium for evaporative cooling system water management, the non-transitory computer readable medium, when read by at least one processor, is configured to execute operations comprising: directing ingress of high purity water at a first amount, where water contains a very low amount of organic and inorganic contamination, less than 20 ppb of TOC and equivalent of greater than 0.02 MΩ·cm of conductive ions by sensors that could include one or more of the following: TOC, conductivity; directing addition of one or more treatment chemicals that could include: corrosion inhibitors, biocides, antiscalants, or dispersants, by sensors that could include one or more of the following: conductivity, ORP, pH; directing usage of the water to absorb heat, which water evaporates to a second amount less than the first amount; and directing replenishment of the second amount of water to the first amount without discarding the one or more treatment chemicals that could include: corrosion inhibitors, biocides, antiscalants, or dispersants.

In an aspect, an apparatus for treating recirculating water in an evaporative cooling system, the apparatus comprising at least one controller having circuitry, which one or more controller is configured to: operatively couple to side stream configured to accommodate water; using one or a combination of a pump and valve in case of a pressurized feedline, direct ingress of a portion of recirculating water from the condenser loop flow via a side stream; using one or a combination of a pump and valve in case of a pressurized feedline, direct side stream water through a filter to remove contaminants that may originate from one or more sources, including air and piping, such that the water meets quality criteria of <5 SDI units and turbidity <2 NTU; and using one or combination of pump or valve in case of a pressurized feedline, direct the filtered side stream water that meets quality criteria to the recirculating water condenser loop.

In an aspect, a non-transitory computer readable medium for treating recirculating water in an evaporative cooling system, the non-transitory computer readable medium, when read by at least one processor, is configured to execute operations comprising: directing ingress of a portion of recirculating water from the condenser loop flow via a side stream; direct side stream water through a filter to remove contaminants so that the water meets quality criteria of <5 SDI units and turbidity <2 NTU, by a sensor that could include one or more of the following: SDI, turbidity, conductivity; and directing the filtered side stream water that meets quality criteria back to the recirculating water condenser loop.

Any of the systems and/or methods disclosed herein may be controlled or performed by a computer system or processor capable of executing program code to perform the steps described herein. For example, system may be a computing system that includes a processing system, storage system, software, communication interface and a user interface. The processing system loads and executes software from the storage system. When executed by the computing system, software module directs the processing system to operate as described in herein in further detail.

The processor or processing system can comprise a microprocessor and other circuitry that retrieves and executes software from storage system. Processing system can be implemented within a single processing device but can also be distributed across multiple processing devices or sub-systems that cooperate in existing program instructions. Examples of processing system include general purpose central processing units, applications specific processors, and logic devices, as well as any other type of processing device, combinations of processing devices, or variations thereof.

The storage system can comprise any storage media readable by processing system, and capable of storing software. The storage system can include volatile and non-volatile, removable and non-removable media implemented in any method or technology for storage of information, such as computer readable instructions, data structures, program modules, or other data. Storage system can be implemented as a single storage device but may also be implemented across multiple storage devices or sub-systems. Storage system can further include additional elements, such a controller capable, of communicating with the processing system.

Examples of storage media include random access memory, read only memory, magnetic discs, optical discs, flash memory, virtual memory, and non-virtual memory, magnetic sets, magnetic tape, magnetic disc storage or other magnetic storage devices, or any other medium which can be used to storage the desired information and that may be accessed by an instruction execution system, as well as any combination or variation thereof, or any other type of storage medium. In some implementations, the store media can be a non-transitory storage media. In some implementations, at least a portion of the storage media may be transitory. It should be understood that in no case is the storage media a propagated signal.

While various embodiments of the present invention have been described above, it should be understood that they have been presented by way of example only, and not limitation. It will be apparent to persons skilled in the relevant art that various changes in form and detail can be made therein without departing from the spirit and scope of the present invention. Thus, the breadth and scope of the present invention should not be limited by any of the above-described exemplary embodiments, but should be defined only in accordance with the following claims and their equivalents.

What is claimed is:

1. A method of water management in an evaporative cooling system comprising: a high temperature element requiring cooling; an evaporative system and water based coolant recirculating between the two, the method comprising:
providing an initial portion of water at a first amount and quality to the evaporative system, wherein the water contains organic and inorganic contamination of less than 20 ppb total organic carbon (TOC) and/or wherein the water contains conductive ions in a concentration resulting in resistivity equal to or greater than 0.02 MΩ·cm;
treating the initial portion of water in the evaporative system with at least one chemical;
causing evaporation of the treated make-up water to reduce the first amount of water to a second amount of water in the evaporative cooling system container; and
adding make-up water to the evaporative cooling system container to increase volume of the water in the container from the second amount to the first amount without discarding substantially any of the treated system water.

2. The method of claim 1, wherein the chemical is a corrosion inhibitor, a biocide, an antiscalant or a dispersant.

3. An apparatus for evaporative cooling system water management, the apparatus comprising:
an evaporative cooling system, a heat source and a water recirculation loop fluidically coupling the evaporative cooling system and the heat source such that the heat source is capable of receiving cooling water supply from the evaporative cooling system via the recirculation loop;
a purified water feed system having an incoming water inlet for receiving water and fluidically coupled to the evaporative cooling system to provide water to the evaporative cooling system;
a chemical treatment system fluidically coupled to the evaporative cooling system to provide at least one of corrosion inhibitors, biocides, antiscalants and/or dispersants to water in the evaporative cooling system; and
a water filtration system comprising at least one of a side-stream and a full-stream filter fluidically coupled to an output of the heat source and an input of the evaporative cooling system, for filtering water from the heat source to remove waste and provide the filtered water to the evaporative cooling system.

4. The apparatus of claim 3, wherein the at least one of the side stream filter and the full stream filter is backwashed using water from an external source to preserve chemicals in the system

5. The apparatus of claim 3, wherein the water has organic and inorganic contamination of less than 20 ppb TOC and/or the water contains conductive ions in a concentration resulting in resistivity equal to or greater than 0.02 MΩ·cm.

6. The apparatus of claim 3, further comprising at least one sensor for sensing at least one of conductivity, oxidation-reduction potential (ORP), pH, silt density index (SDI), turbidity, TOC, and resistivity.

7. The apparatus of claim 3, further comprising at least one sensor or instrument to monitor the concentration of treatment chemicals directly or by measuring concentration of a certain tracing compounds

8. The apparatus of claim 7, wherein at least one of the sensors is in the purified water feed system for measuring at least one of TOC and resistivity.

9. The apparatus of claim 3, further comprising a processor, communicatively coupled to a least one of the purified water feed system, the chemical treatment system and/or the water filtration system for controlling operation of at least of one of the purified water feed system, the chemical treatment system and/or the water filtration system.

10. The apparatus of any of claim 9, wherein the processor is configured to:
direct ingress of the water at a first amount, where the water contains a very low amount of organic and inorganic contamination, less than 20 ppb of TOC or equivalent of greater than 0.02 MΩ·cm of conductive ions;
direct addition of one or more treatment chemicals that could include: corrosion inhibitors, biocides, antiscalants, or dispersants;
direct usage of the water to absorb heat, which dissipates as the water evaporates to a second amount equal or less than the first amount; and
direct replenishment of the second amount of water to the first amount without intentional discarding the one or more treatment chemicals that could include: corrosion inhibitors, biocides, antiscalants, or dispersants.

11. A non-transitory computer readable medium for evaporative cooling system water management, the non-transitory computer readable medium, when read by at least one processor, is configured to execute operations comprising:
directing ingress of water at a first amount to a container, wherein the water contains less than 20 ppb of TOC and/or conductive ions in a concentration resulting in resistivity of greater than or equal to 0.02 MΩ·cm according to sensors measuring one or more of the following: TOC, conductivity;
directing addition of one or more treatment chemicals to the water that could include: corrosion inhibitors, biocides, antiscalants, or dispersants, according to sensors measuring one or more of the following: conductivity, ORP, pH, tracer concentration, silt density index (SDI), turbidity, TOC, and resistivity;
directing usage of the water to absorb heat, which water evaporates to a second amount less than the first amount; and
directing replenishment of the second amount of water to the first amount without discarding the one or more treatment chemicals that could include: corrosion inhibitors, biocides, antiscalants, or dispersants.

12. A method of treating recirculating water in an evaporative cooling system, the method comprising:
filtering the full stream or a diverted portion of recirculating water to remove contaminants such that the water meets quality criteria of <5 SDI units and turbidity <2 NTU; and
returning the filtered stream water that meets quality criteria to the condenser loop flow.

13. The method of claim 12, wherein the contaminants originate from one or more sources, including air and piping.

14. An apparatus for treating recirculating water in an evaporative cooling system having a recirculating water condenser loop and side stream, the apparatus comprising at least one controller having circuitry communicatively coupled to the stream having a filter in a stream flow path, which one or more controller is configured to:
direct ingress of recirculating water from the recirculating water cooling loop;
direct water stream through the filter to remove contaminants such that the water meets quality criteria of <5 SDI units and turbidity <2 NTU; and
direct the filtered stream water that meets quality criteria to the recirculating water condenser loop.
